(19) United States
(12) Patent Application Publication
(10) Pub. No.: US 2023/0248308 A1
(43) Pub. Date: Aug. 10, 2023

(54) SYSTEMS AND METHODS FOR A HAPTIC NEURO-SPATIAL REHABILITATION AND THERAPY DEVICE

(71) Applicants: Arizona Board of Regents on Behalf of Arizona State University, Tempe, AZ (US); Dignity Health, San Francisco, CA (US)

(72) Inventors: Claudio Vignola, Lodi Vecchico (IT); CherryLynne Nethken, Gilbert, AZ (US); Kristin Palmiscno, San Francisco, CA (US); Troy McDaniel, Gilbert, AZ (US); Nikita Joshi, Milpitas, CA (US); Athul Rajeev, Tempe, AZ (US)

(21) Appl. No.: 18/166,073

(22) Filed: Feb. 8, 2023

Related U.S. Application Data
(60) Provisional application No. 63/307,832, filed on Feb. 8, 2022.

(51) Int. Cl. A61B 5/00

(52) U.S. Cl. CPC A61B 5/486; A61B 5/6805; A61B 5/4561; A61B 5/7455; A61B 5/742; A61B 5/4842; A61B 2562/0219

(57) ABSTRACT

Described herein is an assistive medical system that is designed for individuals who are impacted by Midline-Shift Syndrome. Its purpose is to help them reorient and maintain their midline position to improve posture, balance, and other cognitive functions. This is achieved through haptic-feedback mechanisms and visual cues that gently notify the user when their position has deviated from the midline orientation.

### CROSS REFERENCE TO RELATED APPLICATIONS

This is a non-provisional application that claims benefit to U.S. Provisional Application Ser. No. 63/307,832, filed on Feb. 8, 2022, which is herein incorporated by reference in its entirety.

### FIELD

The present disclosure generally relates to rehabilitation devices, and in particular, to a system and associated method for providing correctional postural feedback through a rehabilitation device.

### BACKGROUND

Midline Shift Syndrome (MSS), also known as Pusher Syndrome, is a condition that affects individuals who experience neurological events such as a Traumatic Brain Injury (TBI), Cerebrovascular accident (CVA), Multiple Sclerosis (MS), strokes, etc. MSS causes those affected to lean their body away from the non-paralyzed side on a continual basis due to a change in patients' perception of their body's orientation. This change in perception causes patients to feel that their body posture is upright, whereas, in reality, it is leaning towards the side of the brain lesion. Clinicians trained in rehabilitation techniques who work with these individuals expressed the need for a cost-effective, automated method that can be used to treat Midline Shift Syndrome by helping patients to relearn where their midline position is located, whether rehabilitation sessions be conducted in their medical establishment or in the comfort of the patient's own home. Currently, clinicians have to use manual means for providing haptic feedback to their patients, meaning they use tools such as massage wands and have to physically press such a device onto particular regions of the patient's body as the patient observes themselves in a mirror. This manual technique results in subjective reporting of patient recovery progress.

It is with these observations in mind, among others, that various aspects of the present disclosure were conceived and developed.

### BRIEF DESCRIPTION OF THE DRAWINGS

The present patent or application file contains at least one drawing executed in color. Copies of this patent or patent application publication with color drawing(s) will be provided by the Office upon request and payment of the necessary fee.

FIG. 1 is a simplified illustration showing various electronic components of a haptic neuro-spatial rehabilitation and therapy system;

FIG. 2A is an illustration showing a wearable vest for sensing a midline shift of a patient and providing haptic feedback according to the system of FIG. 1;

FIG. 2B is an illustration of the vest of FIG. 2A with the pads of FIG. 4 attached;

FIG. 3 is an illustration showing IMU placement on the front and back of the wearable vest of FIG. 2A;

FIG. 4 is an illustration showing a front view of a vibration pad of the system of FIG. 1;

FIG. 5 is an illustration showing an isometric view of the vibration pad of FIG. 4;

FIG. 6 is an illustration showing a rear view of the vibration pad of FIG. 4;

FIG. 7 is an illustration showing a neckband sensing a midline shift of a patient and providing haptic feedback according to one example of the system of FIG. 1;

FIG. 8 is an illustration showing IMU placement on the front and back of the neckband of FIG. 7;

FIG. 9 is an illustration showing a smart mirror for visual feedback of a midline shift of a patient according to the system of FIG. 1;

FIGS. 10A-10C are a series of illustrations showing color sequencing by way of feedback displays of the smart mirror of FIG. 9 to provide visual feedback;

FIG. 11 is an electrical schematic showing example electrical components of the wearable vest of FIG. 2A;

FIG. 12 is a photograph showing a plurality of vibrational actuators of the wearable vest of FIG. 2A;

FIG. 13 is an electrical schematic showing the plurality of vibrational actuators in electrical communication with a central processor of the wearable vest of FIG. 2A;

FIG. 14 is a simplified schematic showing a plurality of vibrational actuators in electrical communication with a central processor of the neckband of FIG. 7;

FIG. 15 is a simplified schematic showing a plurality of vibrational actuators in electrical communication with a central processor of the neckband of FIG. 7 in a breadboard testing configuration;

FIG. 16 is a diagram showing a process flow of a midline correction process of the system of FIG. 1;

FIGS. 17A-17E are a series of user interface screenshots of a mobile application which can be used with the system of FIG. 1; and

FIG. 18 is a simplified diagram showing an exemplary computing system which can be used for implementation of aspects of the system of FIG. 1.

Corresponding reference characters indicate corresponding elements among the view of the drawings. The headings used in the figures do not limit the scope of the claims.

### DETAILED DESCRIPTION

Various embodiments of a user-friendly system including at least one device for stroke and other brain injury patients to assist the patients in relearning midline position through a haptic-feedback mechanism are disclosed herein. There are several ongoing therapies that utilize rehabilitation devices under the supervision of skilled therapists, but the objective of this system is to reduce the need for manually applied haptic techniques and improve measurement of patient progress. The system's wearability meets both functional and usability requirements by targeting the torso and neck to provide gentle feedback to the patient. The system includes multiple integrated devices that detect the patient's position in space and provides both visual and haptic cues to the patient and help them reorient back to their midline position.

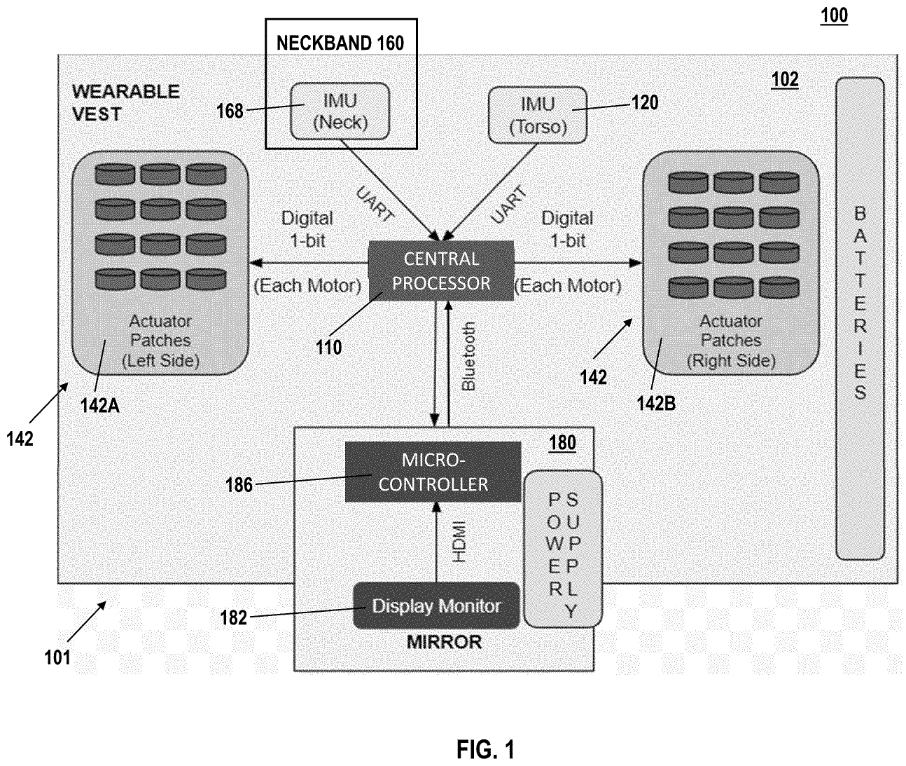

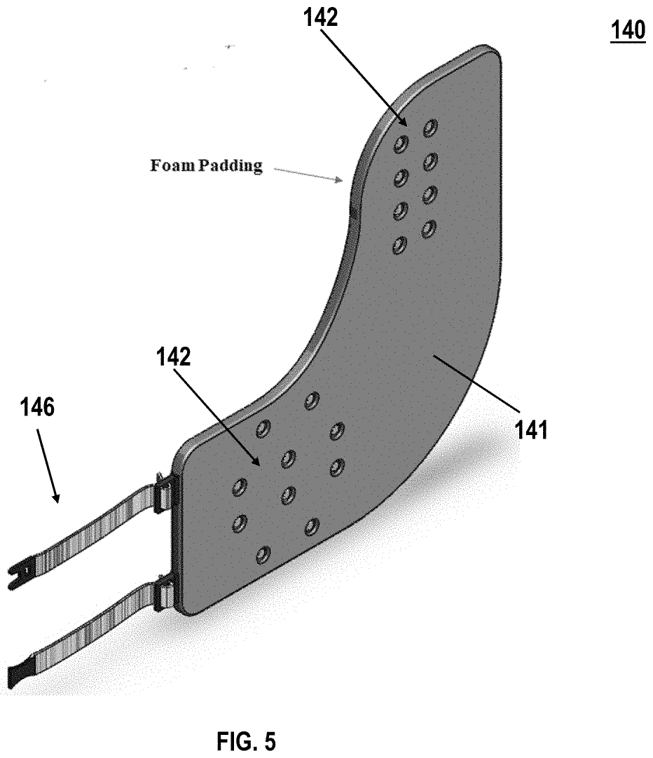

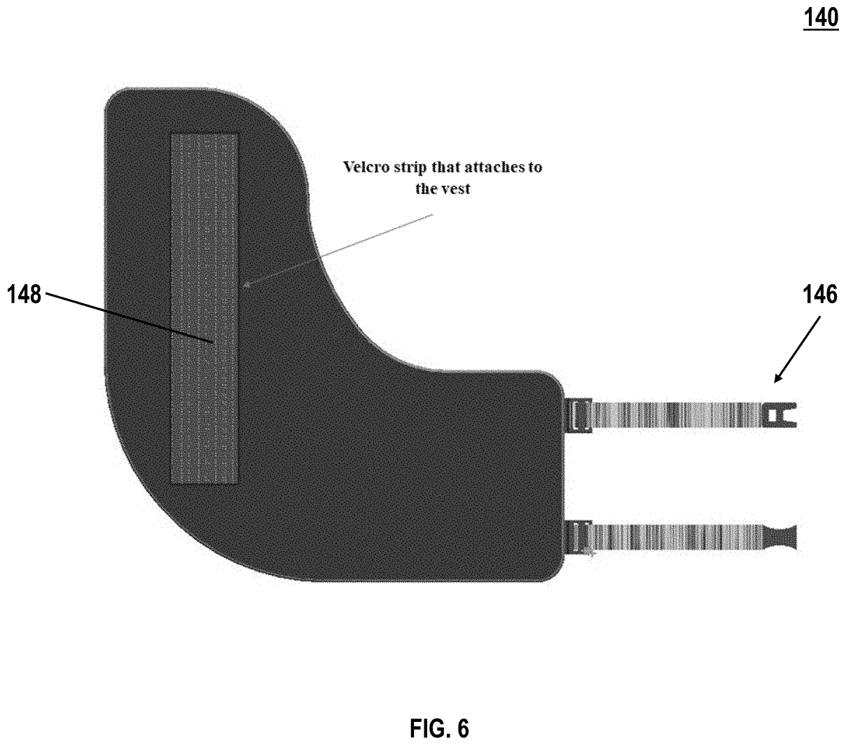

Referring to FIGS. 1-10C, one example of a system 100 for providing correctional postural feedback includes a vest 102 in operative association with a plurality of peripheral devices 101 including a plurality of vibration pads 140, a neckband 160, and a smart mirror 180. In some embodiments, the vest 102 serves as a primary central component of the system 100. The vest 102 includes an electronic inertial measurement unit (IMU) sensor 120 to detect a position of the patient by measuring an angle of the midline shift near the torso region. The vibration pads 140 provide haptic feedback to the shoulders and torso region based on the midline shift measurement detected. The system 100 accommodates improvement of the proprioceptive feedback in the patient by raising the patient's awareness by visual and haptic feedback. In other words, the system 100 uses haptic and visual feedback integrated together to improve the patient's balance. Further, the system 100 improves upon rehabilitation techniques for midline shift and can work with patients that have other symptoms affecting the spatial, visual, and motor response. In some embodiments, the vibration pads 140 include a foam-like padding 144 that incorporates one or more vibration motors 142. These can be easily removable and re-attachable to the vest 102 via a hook-and-loop fastener or other suitable means to adhere to the cleanability requirement of the vest 102.

Since the vest 102 and the vibration pads 140 function together in controlling the midline shift of the upper extremity, a separate component can be used to assess and control the head movements. The neckband 160 is a comfortable soft-robotics wearable that incorporates both an IMU sensor 164 to detect the head position and one or more vibration motors 162 to provide haptic feedback on the neck region. The smart mirror 180 acts as an additional interactive device that provides visual feedback to the patients by communicating with the vest 102 and the neckband 160. The smart mirror 180 can include a microcontroller 186 and a display monitor 182 arranged with one or more feedback displays 184 that can include one or more lights such as LEDs, and can provide color sequencing, e.g., portions of the feedback displays 184 can illuminate in various colors to provide visual cues to aid the patient in shifting back to a midline position.

While products such as smart mirrors are relatively easy to modify for resolving Midline Shift Syndrome (MSS), due to their lack of haptic feedback, they do not act on the muscle memory of the body to provide haptic feedback. For example, the Truweo “Posture Corrector for men and women” is a current alternative that utilizes traditional methods to retrain the body to maintain correct posture. However, when working with MSS patients, forcing the body into position without the proprioception of the mind does not appear to work in recreating the midline perception (meaning without visual cues). Many users of Truweo also shared that the product is uncomfortable and that when the straps move, it leaves marks on the skin.

Other products that have been determined to be more appropriate and less intrusive are the Wearables Upright Go 2, Lumo Lift, and POSDOT “Posture Corrector Digital Sensor Device for Women and Men”. Although the intent of these strapless technologies is to coach their users by utilizing exercises and vibrations—they fail to do so in practice for MSS. This is due to the fact that in able-bodied individuals, it builds reliance on such technologies without having the users learn proper posture and orientation. In the instance of MSS, it is imperative that patients relearn the midline orientation through haptic and visual cues. Additionally, these devices do not take into consideration day-to-day tasks that might require movement, meaning it can incorrectly trigger the warning signals of the device. In individuals with MSS, these present even more of a challenge than a sustainable, long-term solution. Such a device does little to support these individuals to relearn their midline perception and instead can lead to a constant annoyance since they are frequently orienting away from their midline position unlike able-bodied individuals who may only need a reminder now and then to correct their posture. Thus, the disadvantages are the calibration process and the body attachment system.

### Vest

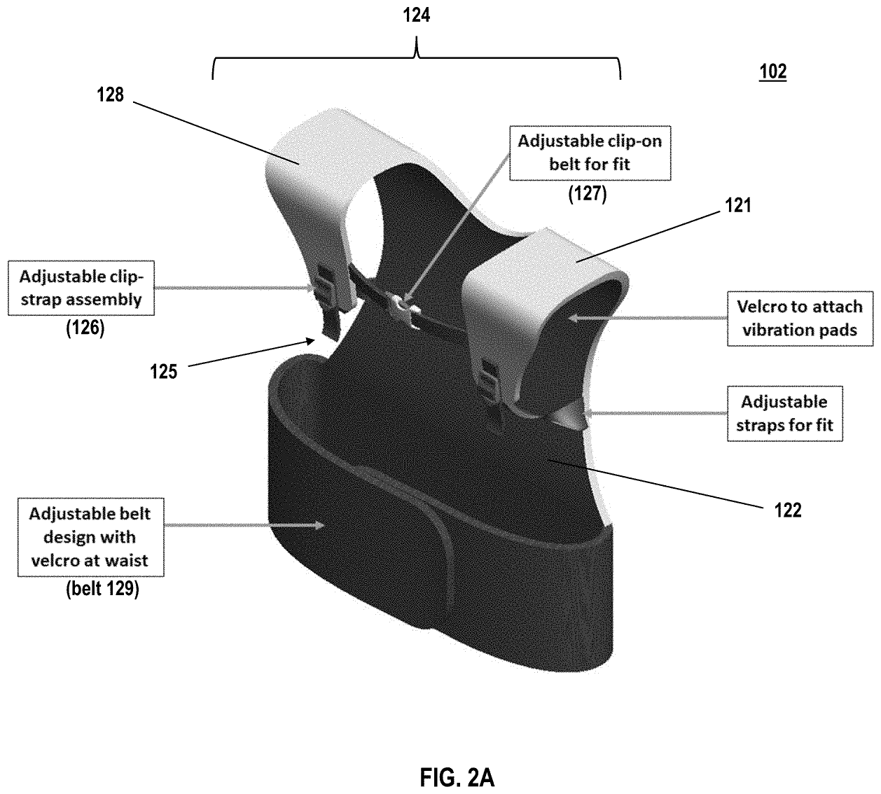

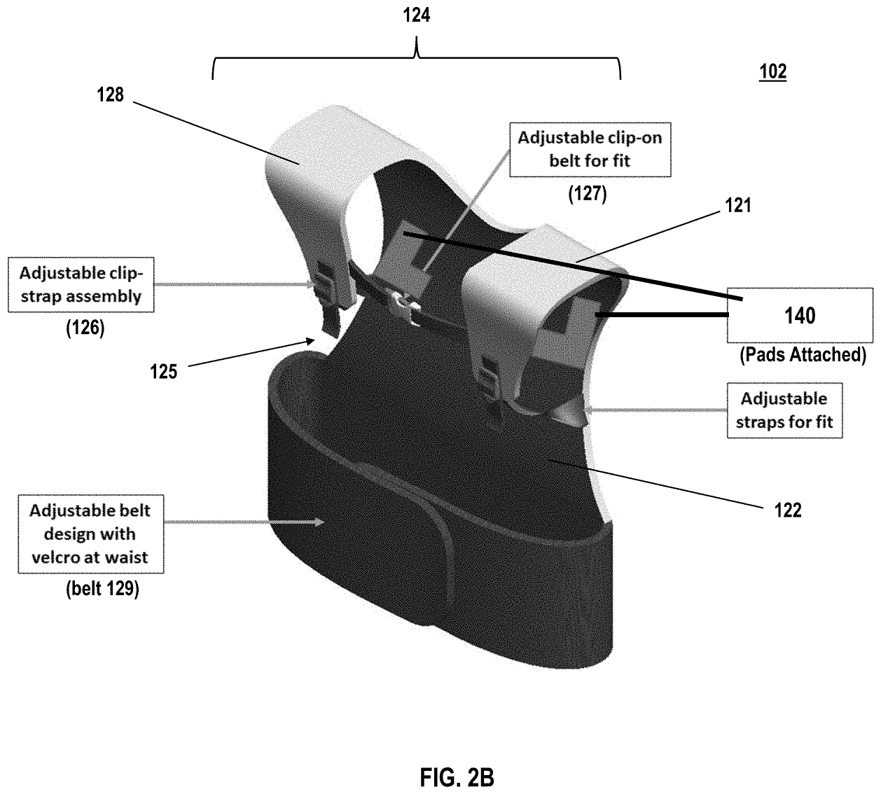

In one example, the wearable vest 102 is lightweight, but at the same time supports the integration of sensors and actuator elements. The wearable vest 102 is also easy for the patient to wear on their body and should feel comfortable when worn on top of clothing. One possible feature of the wearable vest 102 is to be formed by way of a flexible design with features to adjust to different body profiles. FIG. 2A illustrates an example of the wearable vest 102. The wearable vest 102 includes a body 121 with a strap assembly 124 including stretchable straps 125 that are provided on the sides along with an adjustable clip-strap assembly 126 on the front for flexibility. In addition, a clip-on belt 127 can be implemented near the upper chest location to secure one or more front pads 128 of the wearable vest 102 in place. Finally, the wearable vest 102 can be secured onto the mid-waist region with the help of a belt 129 that can include an adjustable belt design and can be fastened using a hook-and-loop fastener or another suitable fastening means.

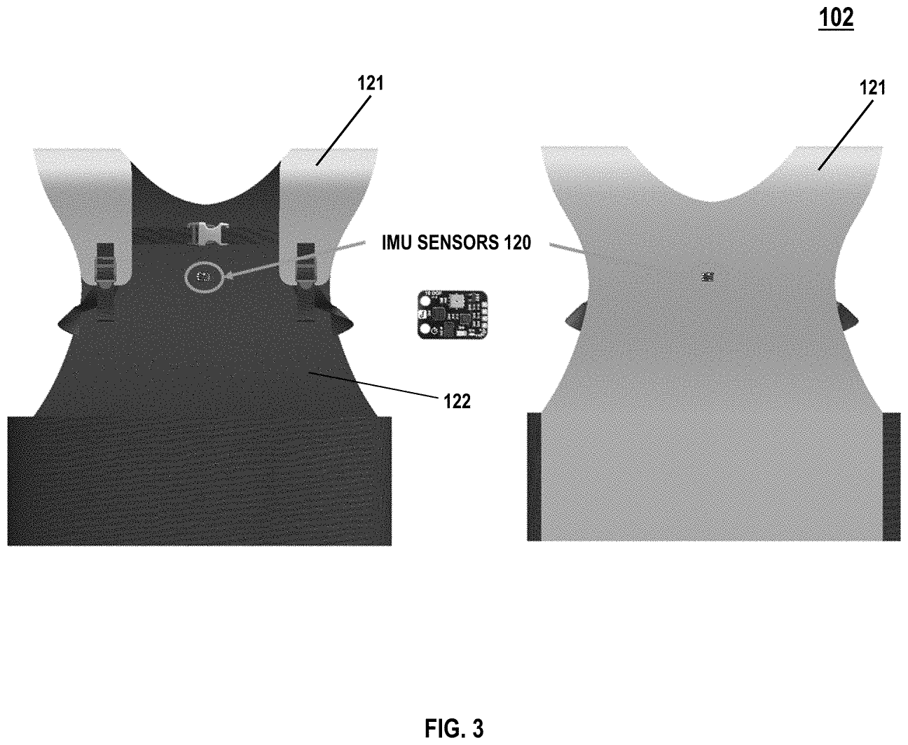

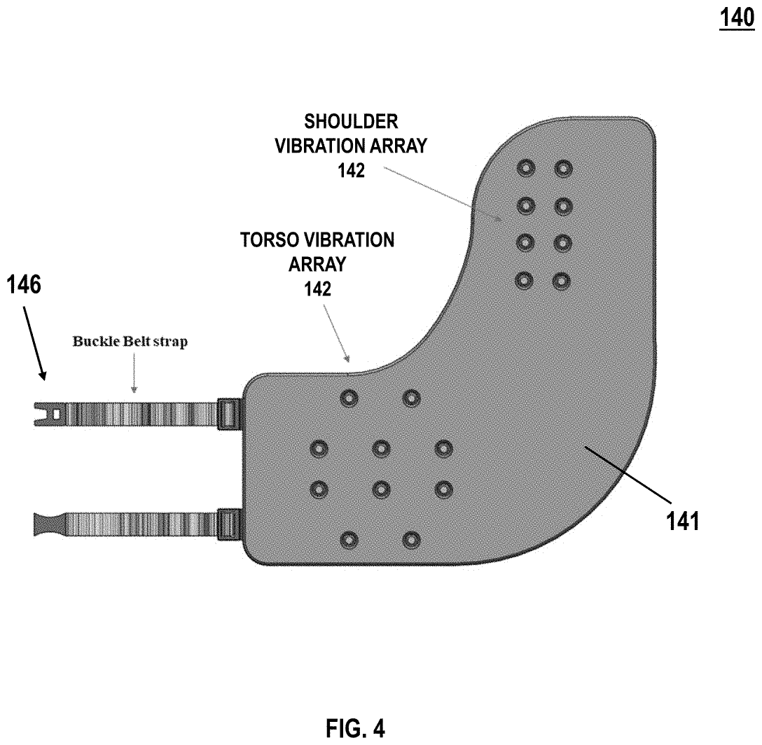

Referring to FIG. 2B, another important aspect of the wearable vest 102 can include integration of the one or more vibration pads 140 (example details of pads 140 shown in FIG. 4) designed separately and easily to attach onto the wearable vest 102 for improved usability. Therefore, an interior face 122 of the wearable vest 102, indicated in blue color in the figure, can be provided with one side of a hook-and-loop adhesive to fasten the two vibration pads 140 (which are provided with an opposite side of a hook-and-loop adhesive) on either side of the vest. In FIG. 3, one example ideal location for the integration of one or more IMU sensors 120 is shown. As depicted, the IMU sensors 120 can be placed in the central and slightly towards the upper half of the torso region. In some embodiments, two IMU sensors 120 are incorporated into the wearable vest 102, one on each side of the rear face of the wearable vest 102. This can improve the accuracy of sensor data acquired from the IMU sensors 120 by canceling any noise that may exist in the environment and indicate an average value of different outputs from the IMU sensors 120.

### Vibration Pads

The vibration pads 140 provide passive feedback to the patient when the orientation of their body deviates from their midline position. In some embodiments, the wearable vest 102 includes two vibration pads 140 removably coupled to the wearable vest 102, each vibration pad 140 defining a body 141 with a hook-and-loop fastener 148 (or another suitable fastener such as snaps) side. Each pad has a circular array of vibration motors 142 placed on the side of the torso right below where the sternum of the ribcage is located and another rectangular array of vibration motors 142 located near the corner shoulder region as seen in FIGS. 4-6.

The locations of the vibration motors 142 were selected as the sensation of touch is relatively higher compared to other body parts in stroke patients. The vibration pads 140 can also be filled with foam to reduce the effect of the vibration motors 142 on the sensitivity of the IMU sensors 120 and also to give a snug fit for the patients. The left and right sides of the vibration pads 140 are joined together at the torso region above the waistline using buckle belt straps 146. Vibration pads 140 can be removable from the vest 102 so that the vest 102 can be washed and reused after therapy sessions.

### Neckband

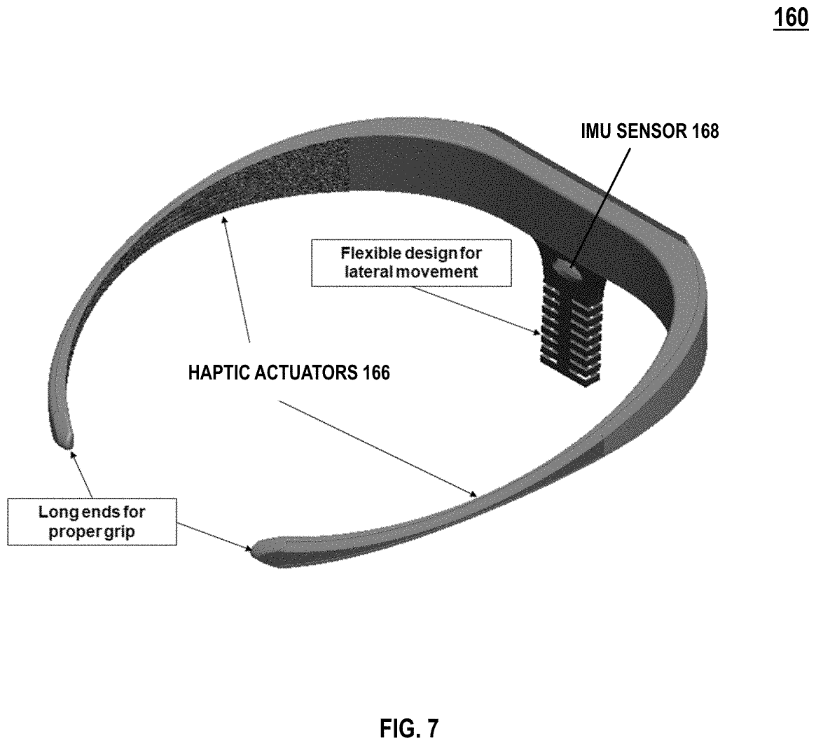

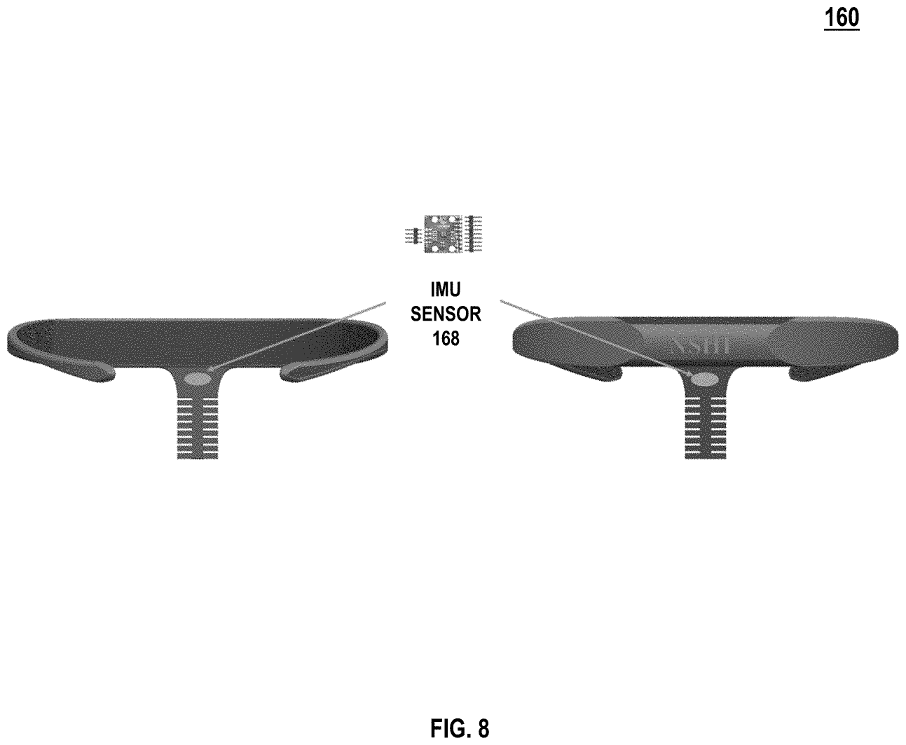

Although the vest 102 and vibration pads 140 can cover the torso area, they do not take into consideration neck movement during the midline shift. However, the system 100 can also track the neck orientation. For example, the neckband 160 is intended to have a cozy fit and provide proper grip around the neck. In some embodiments, the neckband 160 provides a comfortable flexible lateral movement of the head. A flexible rubber-based material may be used for the spinal element in the neckband 160 that is shown in FIG. 7. As shown, haptic actuators 166 are also placed inside the neckband 160 around the left and right hand of the neckband 160 to provide gentle haptic feedback. The haptic actuators 166 provide vibration whenever the orientation of the neck exceeds a threshold limit for deviation from the midline. The orientation of the neck is measured by an IMU sensor 168 located at the back of the neckband shown in FIG. 8.

### Smart Mirror

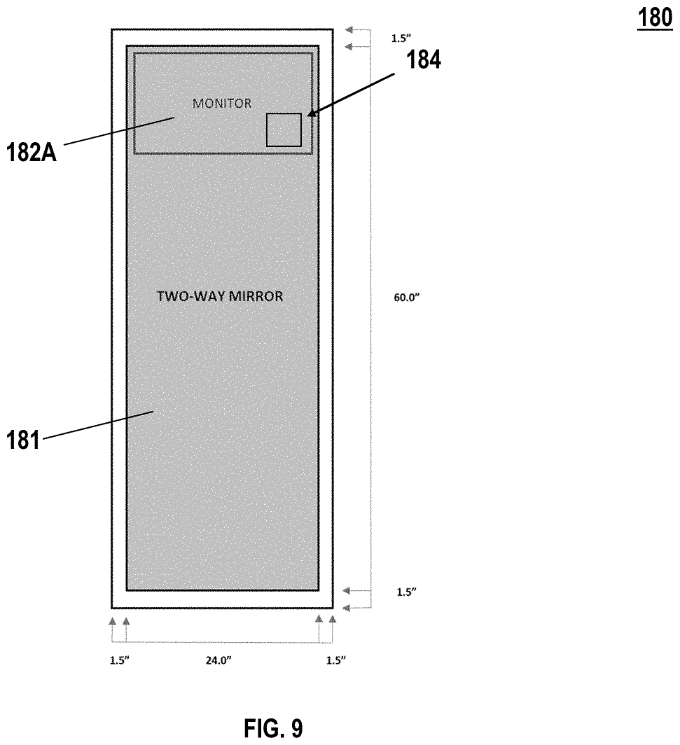

The hardware design of the smart mirror 180 includes a two-way acrylic sheet in which the patient will be able to see what is displayed on an integrated screen as well as their reflection. In some embodiments, a frame of the smart mirror 180 can include a polymer plastic strip with squared corners, although other variations are considered for cosmetic purposes. In some embodiments, the smart mirror 180 can include a stand base for the smart mirror 180 in instances where the therapist or patient cannot prop the smart mirror 180 against a wall. A monitor 182A that displays orientation can be mounted on the top of a two-way mirror 181 as shown in FIG. 9.

The smart mirror 180 can act as an additional aide for providing visual cues to the patient in shifting back to the midline position. Hence, the smart mirror 180 provides a visual feedback system. The smart mirror 180 includes a microcontroller 186 (FIG. 1; and is in some embodiments a Raspberry Pi), the monitor 182A mounted behind the two-way mirror 181, one or more feedback displays 184, along with other supporting components including an HDMI adapter, a VGA adapter, 5.1V and 3A USB-C power adapter, and a frame assembly. The vest 102 and neckband 160 can communicate with the microcontroller 186 of the smart mirror 180 via Wi-Fi or Bluetooth so when the patient is in front of the smart mirror 180 during treatment, the IMU sensors 120 of the vest 102 are operable to detect the change of their movement at the neck or the torso and display their current orientation on the monitor 182A of the smart mirror 180. While the vibration pads 140 provide the haptic feedback to the patient, the smart mirror 180 is designed to display the tilted angle of the body and the direction with the assistance of the IMU sensor. FIG. 9 provides a drawing of an example (non-limiting) basic layout for the smart mirror 180.

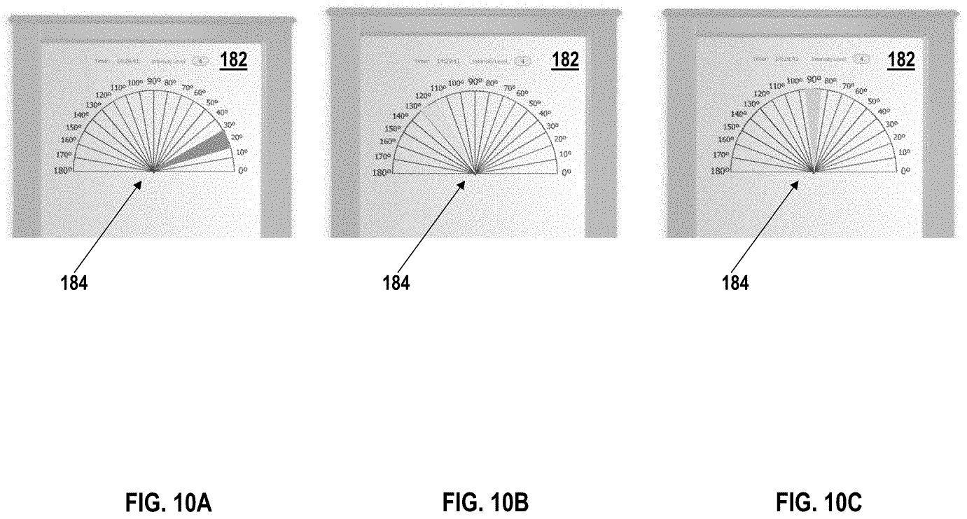

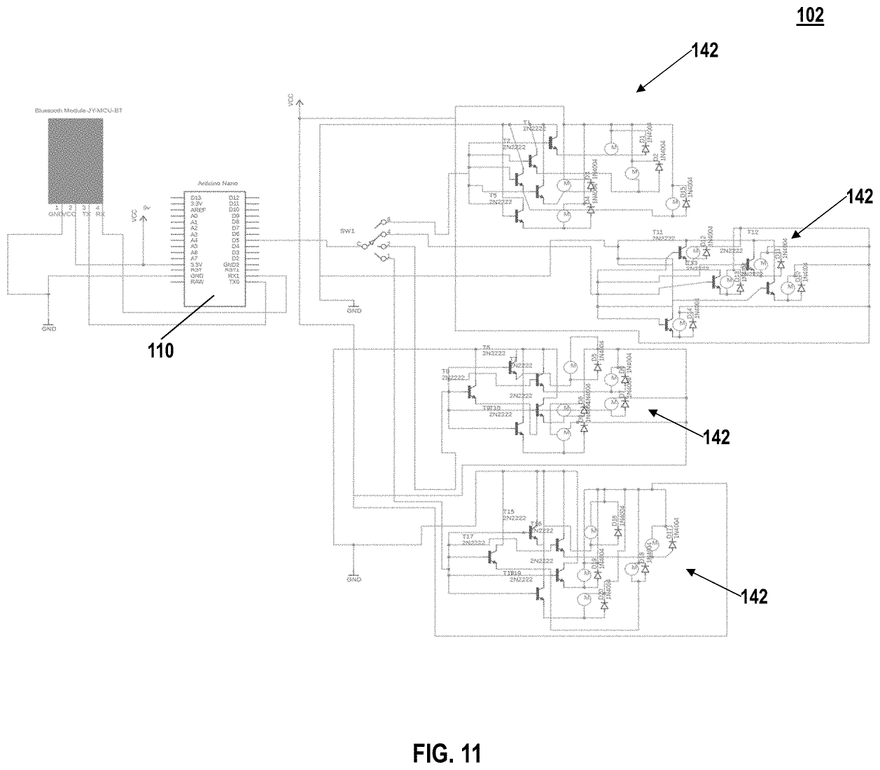

The smart mirror 180 indicates a shift in position from the midline as a means for providing visual feedback to the patient. As shown in FIGS. 10A-10C, when the patient is within the midline range, the monitor 182A displayed through the smart mirror 180 will show an indication that the patient is within the midline range, for instance by showing a green color from the one or more feedback displays 184 (FIG. 10C). On the other hand, when the patient shifts considerably in either direction, the monitor 182A can show an indication of the shift, for instance by showing a red color from the one or more feedback displays 184 (FIG. 10A). If the patient deviates slightly from the midline, the monitor 182A can indicate a slight deviation by showing a yellow color from the one or more feedback displays 184 (FIG. 10B). With the aid of this visual feedback, the patient can attempt to move back to the original posture. In addition to the visual aid, the smart mirror 180 can also provide audio feedback for improved interactivity and accessibility.

### Control System/Electronics

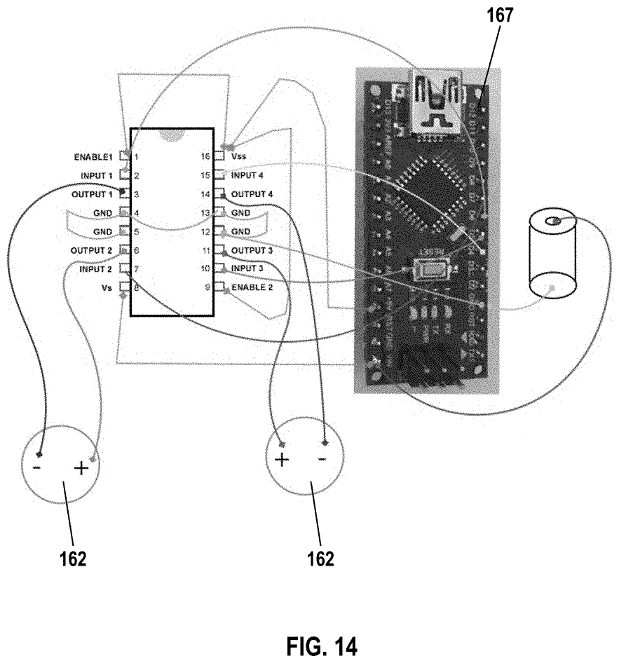

Electronics play a role in the overall operability of the system 100. FIG. 1 provides a simplified visual representation of various integrated components of the system 100 including a central processor 110. As shown, the wearable vest 102 includes IMU sensor 120 and the neckband 160 includes IMU sensor 168 that provides sensor data to the central processor 110. The sensor data from the IMU sensors 120 and 168 is interpreted through an application executed by the central processor 110 to determine if an orientation of the patient is shifted away from midline. When this occurs, the central processor 110 can send one or more pulse-width modulation (PWM) signals to the one or more vibration motors 142 and 162 (where vibration motors 142A and 142B designate left and right arrays shown in FIG. 1 with vibration motors 142 designating all vibration motors 142 located along the wearable vest 102 in FIG. 11; and where vibration motors 162 are located in the neckband 160 of FIG. 14) causing them to vibrate and initiate a haptic feedback sequence for the patient. The sensor data from the IMU sensors 120 and 164 can also be sent from the central processor 110 to the microcontroller 186 of the smart mirror 180 via Bluetooth communication protocols. Power to the wearable vest 102 can be provided through batteries and power to the smart mirror 180 can be provided through a wall outlet. The microcontroller 186 can use a 5.1V and 3A USB-C power adapter and the monitor 182A can use a standard AC power cable.

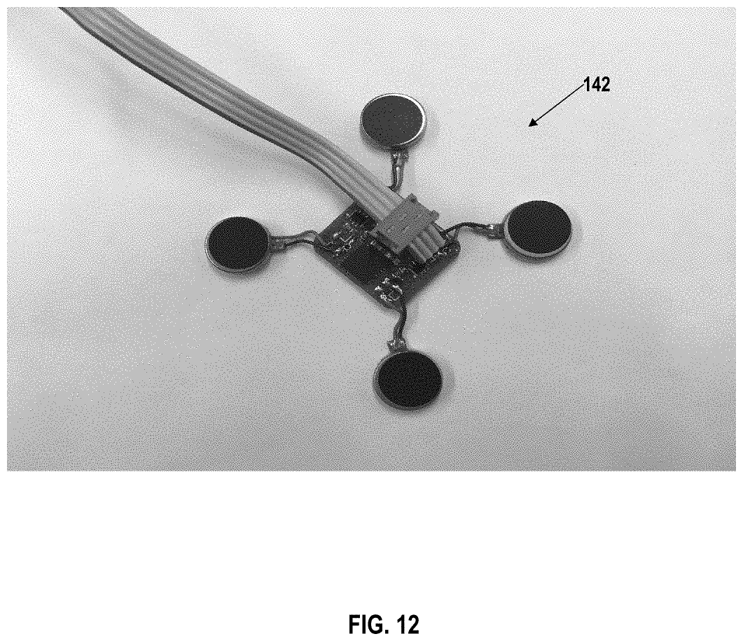

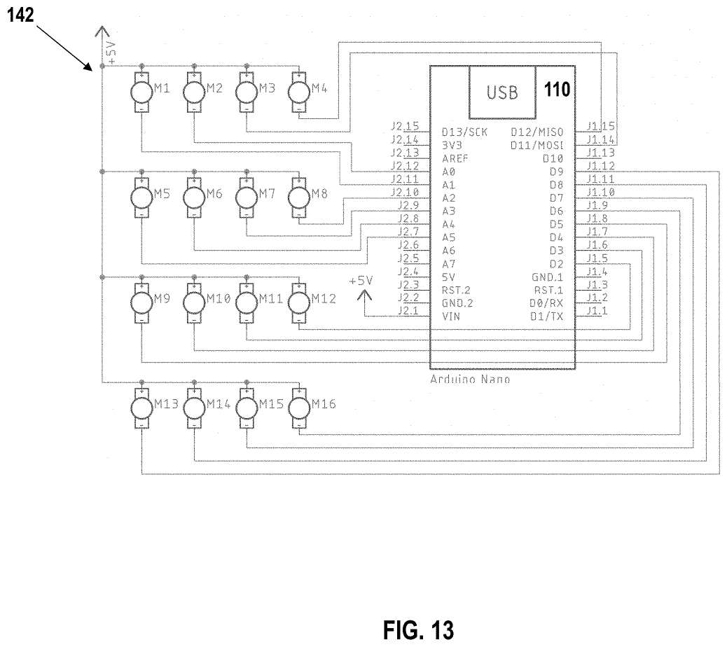

In one embodiment of the wearable vest 102, a total of 20 coin-sized vibration motors 142 can be used, which turn on (1=HIGH) and off (0=LOW). The number of vibration motors 142 can be defined according to the amount of vibration that would theoretically be required for effectively supplying adequate haptic feedback to the patient. However, this requires trials and testing before confirming the exact number of vibration motors 142 that should be used on each side of the wearable vest 102. Trials were not conducted due to COVID-19, meaning human trials were not possible at the time to determine the exact number of motors required for the wearable vest 102. FIG. 11 is an electrical schematic showing a haptic feedback mechanism of the wearable vest 102 using twenty vibration motors 142. In one implementation, four motor packs with five vibration motors 142 each would be suitable for operation in this configuration. In this case, a simple rotary switch can be used to toggle between the motor packs shown in FIGS. 12 and 13. The circuit can be programmed accordingly for turning the motor packs (with the five vibration motors 142 in each packet) on or off.

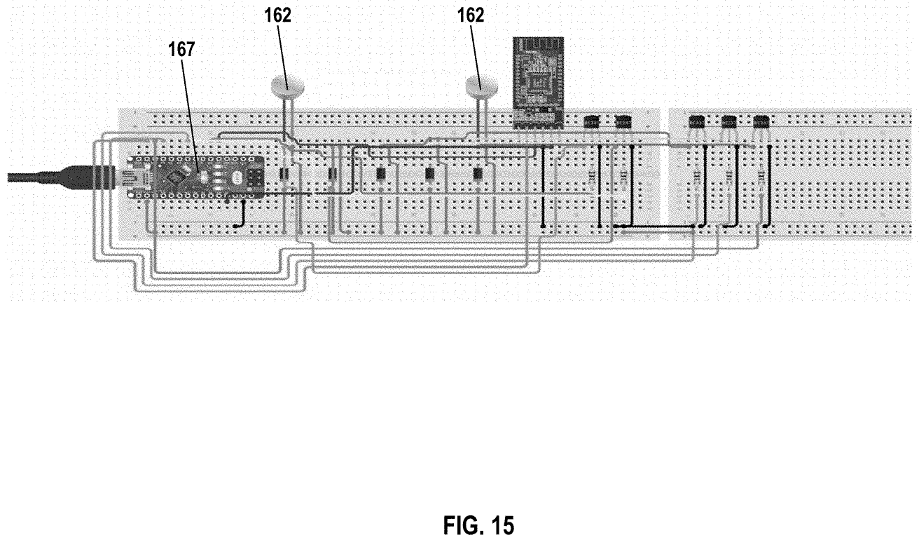

In one embodiment of the neckband 160 (hardware shown in FIG. 14), the neckband 160 includes a controller 167 (which is in some embodiments an Arduino Nano-33-BLE controller, which has BLE 5.0 connectivity with an integrated NINA-b3 (nRF52840) chip, along with 1 Mb flash and 256 KB spam memory). The controller 167 is compact and lightweight in order to fit within the neckband 160 and provides better interconnection between the vibration motors 162 and the rest of the system 100. An electrical schematic (not shown) would be very similar to that of the wearable vest 102, except in some embodiments the neckband 160 includes fewer vibration motors 162. The controller 167 is responsible for driving the vibration motors 162 to vibrate in the required direction with given power. FIG. 15 shows an adapted version of the electrical components of the neckband 160 using a breadboard setup.

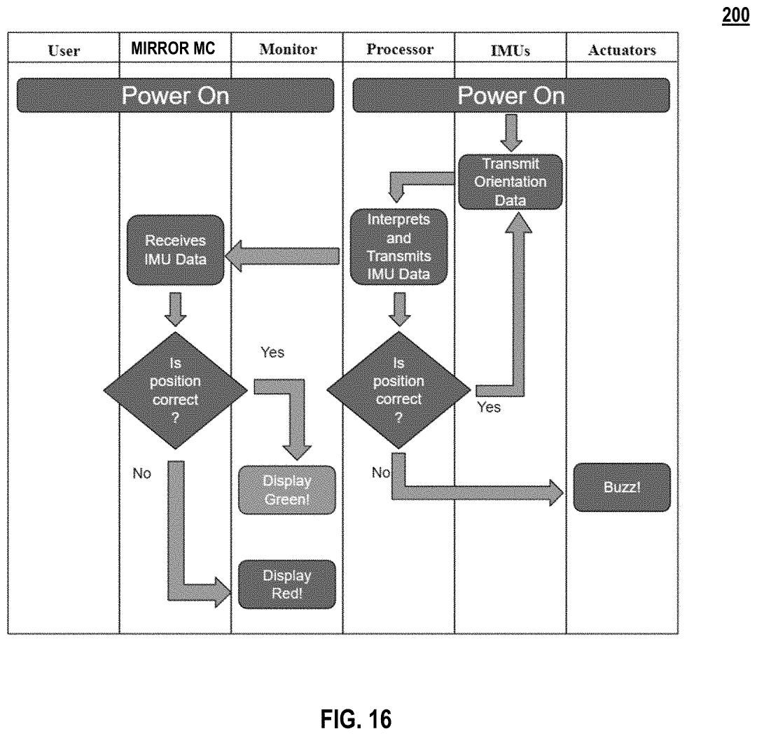

Software application flow and control can be handled by both the microcontroller 186 of the smart mirror 180 and the central processor 110 of the system 100 and is illustrated in process flow 200 of FIG. 16. After the user powers on the system 100, the IMU sensors 120 and 164 of the wearable vest 102 and the neckband 160 can begin transmitting sensor data to the central processor 110. The central processor 110 determines if the patient has reached their correct midline based upon one or more degree angles received as sensor data from the IMU sensors 120 and 164. If the correct position is reached, no haptic feedback will be provided to the patient. However, if the patient has shifted away from their midline, the central processor 110 sends signals to the one or more vibration motors 142 and 162 to begin vibrating. Through the use of software threading, the central processor 110 can simultaneously send the sensor data from the IMU sensors 120 and 164 to the microcontroller 186 of the smart mirror 180. Software on the microcontroller 186 then determines if the patient is in the correct midline and displays either a red, yellow, or green color sequence on the smart mirror 180 based upon their position in space. A visual depiction of this process is provided in FIG. 16.

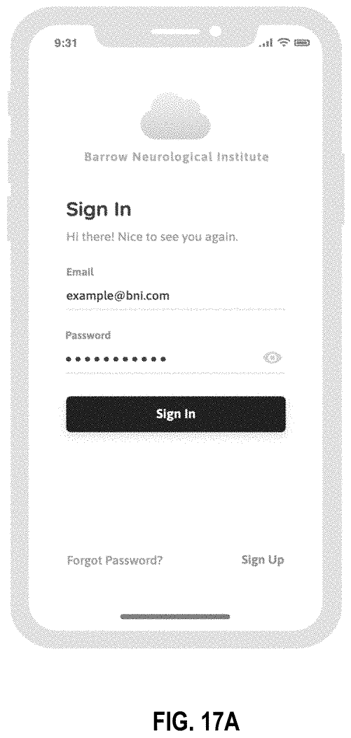

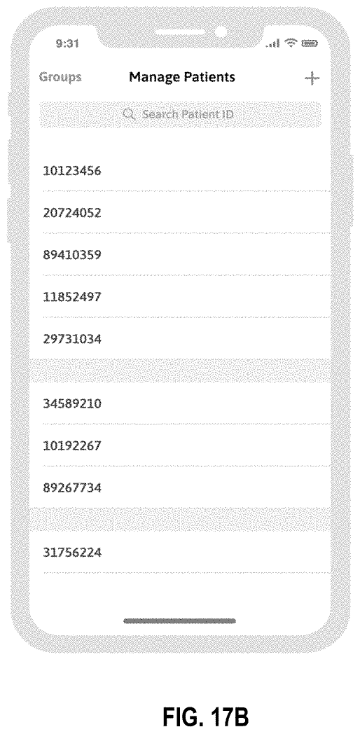

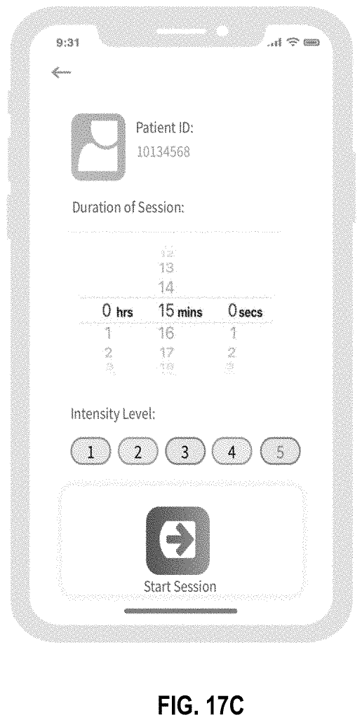

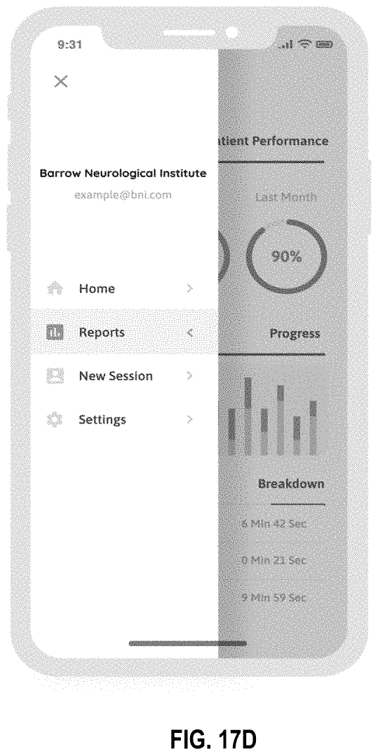

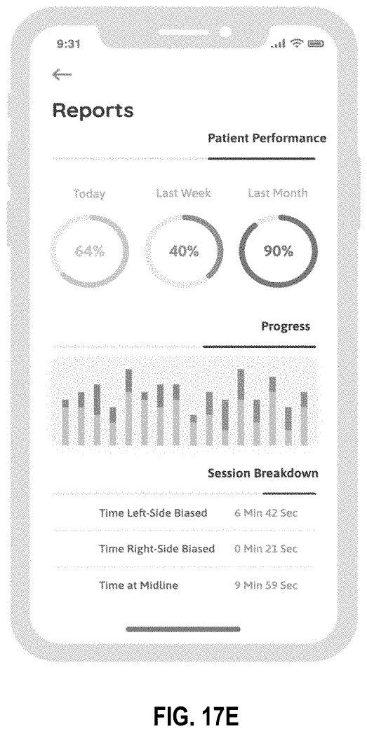

The system 100 can further include a mobile application that can be used to monitor and record patient progress. This reporting system enables clinicians and patients to more accurately track recovery progress, a key component which has inhibited insurance companies from paying for treatment with the current manual techniques that are used. One of a mobile application for the device can be observed in FIGS. 17A-E below. The mobile application includes a sign-in for the clinician, a means to search for patients using unique identifiers for patient privacy, a screen to set training session parameters, and the ability to pull training session reports.

### Computer-Implemented System

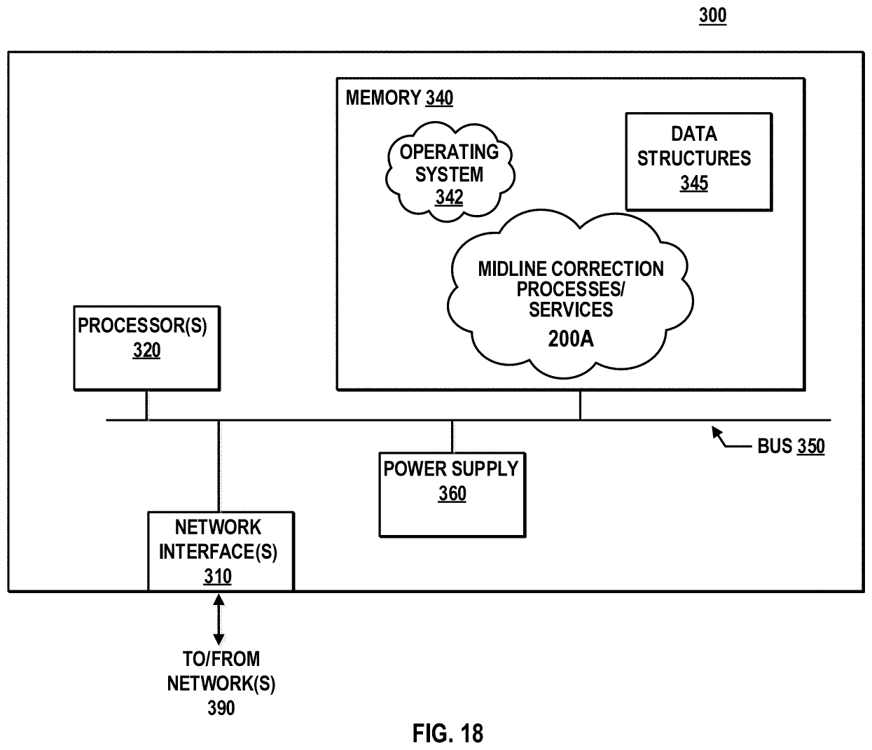

FIG. 18 is a schematic block diagram of an example device 300 that may be used with one or more embodiments described herein, e.g., as a component of system 100 and/or as computing device 104 shown in FIG. 1.

Device 300 comprises one or more network interfaces 310 (e.g., wired, wireless, PLC, etc.), at least one processor 320, and a memory 340 interconnected by a system bus 350, as well as a power supply 360 (e.g., battery, plug-in, etc.).

Network interface(s) 310 include the mechanical, electrical, and signaling circuitry for communicating data over the communication links coupled to a communication network. Network interfaces 310 are configured to transmit and/or receive data using a variety of different communication protocols. As illustrated, the box representing network interfaces 310 is shown for simplicity, and it is appreciated that such interfaces may represent different types of network connections such as wireless and wired (physical) connections. Network interfaces 310 are shown separately from power supply 360, however it is appreciated that the interfaces that support PLC protocols may communicate through power supply 360 and/or may be an integral component coupled to power supply 360.

Memory 340 includes a plurality of storage locations that are addressable by processor 320 and network interfaces 310 for storing software programs and data structures associated with the embodiments described herein. In some embodiments, device 300 may have limited memory or no memory (e.g., no memory for storage other than for programs/processes operating on the device and associated caches).

Processor 320 comprises hardware elements or logic adapted to execute the software programs (e.g., instructions) and manipulate data structures 345. An operating system 342, portions of which are typically resident in memory 340 and executed by the processor, functionally organizes device 300 by, inter alia, invoking operations in support of software processes and/or services executing on the device. These software processes and/or services may include midline correction processes/services 200A, which may include the same or similar functions as the process flow 200 described herein. Note that while midline correction processes/services 200A is illustrated in centralized memory 340, alternative embodiments provide for the process to be operated within the network interfaces 310, such as a component of a MAC layer, and/or as part of a distributed computing network environment.

It will be apparent to those skilled in the art that other processor and memory types, including various computer-readable media, may be used to store and execute program instructions pertaining to the techniques described herein. Also, while the description illustrates various processes, it is expressly contemplated that various processes may be embodied as modules or engines configured to operate in accordance with the techniques herein (e.g., according to the functionality of a similar process). In this context, the term module and engine may be interchangeable. In general, the term module or engine refers to model or an organization of interrelated software components/functions. Further, while the midline correction processes/services 200A is shown as a standalone process, those skilled in the art will appreciate that this process may be executed as a routine or module within other processes.

It should be understood from the foregoing that, while particular embodiments have been illustrated and described, various modifications can be made thereto without departing from the spirit and scope of the invention as will be apparent to those skilled in the art. Such changes and modifications are within the scope and teachings of this invention as defined in the claims appended hereto.

What is claimed is:

1. A system, comprising:
a wearable vest including:
one or more inertial measurement units located along a midline of the wearable vest; and
one or more vibration motors configured to provide haptic feedback to a user;
a neckband including:
one or more inertial measurement units located along a midline of the neckband; and
one or more vibration motors configured to provide haptic feedback to a user; and
a central processor in communication with the wearable vest, the neckband and a memory, the memory including instructions which, when executed, cause the central processor to:
determine an angle of a midline shift based on sensor data provided by the one or more inertial measurement units located along the midline of the wearable vest and of the neckband; and
provide a signal to the one or more vibration motors of the wearable vest and of the neckband based on the angle of the midline shift to provide haptic feedback to the user.

2. The system of claim 1, further comprising:
a mirror including a monitor in communication with the central processor, the monitor configured to provide visual feedback to the user.

3. The system of claim 2, wherein the visual feedback is provided on the monitor using one or more LED indicators.

4. The system of claim 2, wherein the memory includes instructions which, when executed, further cause the central processor to:
indicate, by the monitor, a severity of the midline shift based on the angle of the midline shift as determined using the sensor data.

5. The system of claim 1, wherein the one or more vibration motors are positioned along a vibration pad, and the vibration pad is removably coupled to the wearable vest.

6. A system, comprising:
a wearable vest including:
one or more inertial measurement units located along a midline of the wearable vest; and
one or more vibration motors configured to provide haptic feedback to a user; and
a processor in communication with the wearable vest and a memory, the memory including instructions which, when executed, cause the processor to:
determine an angle of a midline shift based on sensor data provided by the one or more inertial measurement units located along the midline of the wearable vest; and
provide a signal to the one or more vibration motors of the wearable vest based on the angle of the midline shift to provide haptic feedback to the user.

7. The system of claim 6, further comprising:
a mirror including a monitor in communication with the processor, the monitor configured to provide visual feedback to the user.

8. The system, of claim 7, wherein the visual feedback is provided on the monitor using one or more LED indicators.

9. The system of claim 7, wherein the memory includes instructions which, when executed, further cause the processor to:
displaying, by the monitor, a visual representation of the midline shift based on the angle of midline shift.

10. The system of claim 7, wherein the memory includes instructions which, when executed, further cause the processor to:
indicate, by the monitor, a severity of the midline shift based on the angle of the midline shift as determined using the sensor data.

11. The system of claim 6, further comprising:
a neckband including:
one or more inertial measurement units located along a midline of the neckband; and
one or more vibration motors configured to provide haptic feedback to a user.

12. The system of claim 11, wherein the memory includes instructions which, when executed, further cause the processor to:
determine an angle of a midline shift based on sensor data provided by the one or more inertial measurement units located along the midline of the neckband; and
provide a signal to the one or more vibration motors of the neckband based on the angle of the midline shift to provide haptic feedback to the user.

13. The system of claim 6, wherein the one or more vibration motors are positioned along a vibration pad, wherein the vibration pad is removably coupled to the wearable vest.

14. A method of providing midline shift feedback to a patient, the method comprising:
receiving sensor data from one or more inertial measurement units located along a wearable vest;
calculating, based on the sensor data received from one or more inertial measurement units, an angle of a midline shift of the one or more inertial measurement units; and
providing a signal to one or more vibration motors of the wearable vest based on the angle of the midline shift to provide haptic feedback.

15. The method of claim 14, further comprising:
receiving sensor data from one or more inertial measurement units located along a midline of a neckband.

16. The method of claim 15, further comprising:
providing a signal to the one or more vibration motors of the neckband based on the angle of the midline shift to provide haptic.

17. The method of claim 14, wherein the angle of midline shift is representative of a deviation of alignment of the midline of the wearable vest with a midline position.

18. The method of claim 14, further comprising:
displaying, by a monitor, a visual representation of the midline shift based on the angle of midline shift.

19. The method of claim 18, wherein the monitor is mounted within a mirror.

20. The method of claim 18, wherein the monitor is configured to indicate a severity of the midline shift based on the angle of the midline shift as determined using the sensor data.
